(19) United States
(12) Patent Application Publication
(10) Pub. No.: US 2023/0054941 A1
(43) Pub. Date: Feb. 23, 2023

(54) INHIBITION OF DENND5B EXPRESSION FOR TREATING HEPATIC STEATOSIS

(71) Applicant: University of Kentucky Research Foundation, Lexington, KY (US)

(72) Inventor: Scott Gordon, Lexington, KY (US)

(21) Appl. No.: 17/792,388

(22) PCT Filed: Jan. 13, 2021
(86) PCT No.: PCT/US21/13293

§ 371 Date: Jul. 12, 2022

Related U.S. Application Data
(60) Provisional application No. 62/960,485, filed on Jan. 13, 2020.
(60) Provisional application No. 63/032,080, filed on May 29, 2020.

(51) Int. Cl. C12N 15/113; C12N 9/22; C12N 15/11; A61P 1/16

(52) U.S. Cl. CPC C12N 15/113; C12N 9/22; C12N 15/11; A61P 1/16; C12N 2310/11; C12N 2310/141; C12N 2310/14; C12N 2310/3231

(57) ABSTRACT

Methods of treating hepatic steatosis involve administering to a subject a DENND5B inhibitor, thereby reducing the expression of and/or activity of DENND5B in liver of the subject. The DENND5B inhibitor can include antisense oligonucleotide (ASO), CRISPR interference (CRISPRi), miRNA, siRNA, locked nucleic acid (LNA) nucleotides, or a combination thereof.

### RELATED APPLICATIONS

This application claims priority from U.S. Provisional Application Ser. No. 62/960,485, filed Jan. 13, 2020, and 63/032,080, filed May 29, 2020 the entire disclosures of which are incorporated herein by this reference.

### TECHNICAL FIELD

The presently-disclosed subject matter generally relates to controlling absorption of and/or intracellular accumulation of triglycerides and lipids. In particular, certain embodiments of the presently-disclosed subject matter relate to methods of inhibiting DENND5B, thereby controlling absorption of and/or intracellular accumulation of triglycerides and lipids. As disclosed herein, in some embodiments the methods of inhibiting DENND5B can be useful for treating hepatic steatosis, which involves accumulation of triglycerides and lipids in liver tissue.

### INTRODUCTION

Hepatic steatosis, also known as hepatic lipid accumulation or fatty liver disease, is a condition that involves intracellular accumulation of triglycerides and lipids in liver tissue. Etiology can be used to classify hepatic steatosis, which can occur from increased intake of exogenous fat, reduction in the disposal of fat, and/or increased production of endogenous fatty acids.

In some cases, hepatic steatosis can be induced by diet and/or ethanol use. In this regard, the condition can be identified as diet-induced fatty liver disease, non-alcohol-induced fatty liver disease, or alcohol-induced fatty liver disease. Subjects who consume alcohol in large quantities and/or who suffer from obesity are at risk for hepatic steatosis.

Hepatic steatosis affects about 25% of the world's population and an even higher percentage within the United States. Diet-induced fatty liver disease or non-alcohol-induced fatty liver disease is found in obese subjects and is often associated with type II diabetes. Lipid peroxidation of excess fatty acids can create inflammation and steatohepatitis and, for many, progression of this disease will lead to severe liver damage and cirrhosis, necessitating a liver transplant.

Accordingly, there is a significant need for treatments for this condition; however, effective targets and treatments have not yet been identified and there remains no FDA-approved treatment for hepatic steatosis.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes methods of controlling absorption of and/or intracellular accumulation of triglycerides and lipids. Some embodiments of the presently-disclosed subject matter relate to methods of inhibiting DENND5B, thereby controlling absorption of and/or intracellular accumulation of triglycerides and lipids. Some embodiments of the presently disclosed subject matter include reducing lipid accumulation in the liver. Some embodiments of the presently disclosed subject matter include reducing lipid accumulation in the intestine. The presently-disclosed subject matter further includes methods of treating hepatic steatosis in a subject in need thereof or at risk therefore.

The presently-disclosed subject matter includes a method of treating hepatic steatosis, which involves identifying a subject having or at risk of developing hepatic steatosis, and administering to the subject a DENND5B inhibitor, thereby reducing the expression of and/or activity of DENND5B in the subject.

In some embodiments, the DENND5B inhibitor comprises antisense oligonucleotide (ASO), CRISPR interference (CRISPRᵢ), miRNA, siRNA, locked nucleic acid (LNA) nucleotides, or a combination thereof. As will be recognized by one of ordinary skill in the art, inhibitors that operate using nucleotide interference can make use of a nucleotide sequence or a guide antisense strand that is designed in in view of the sequence of the target gene of interest. In the presently-disclosed subject matter, the target gene of interest is DENND5B, the nucleotide sequence of which is provided in SEQ ID NO: 1.

In some embodiments, the DENND5B inhibitor comprises a guide antisense strand consisting of about 10 to about 30 nucleotides. In some embodiments, the DENND5B inhibitor comprises a guide antisense strand consisting of about 10 to about 30 consecutive nucleotides of SEQ ID NO: 1, or a complement thereof. In some embodiments, the DENND5B inhibitor comprises a guide antisense strand consisting of about 10 to about 30 consecutive nucleotides of the first 2216 nucleotides of SEQ ID NO: 1, or a complement thereof.

Some embodiments of the presently-disclosed subject matter involve targeting the reducing the expression of and/or activity of DENND5B to the liver using a targeting moiety. In this regard, the DENND5B inhibitor can include a moiety for directing the inhibitor to the liver. Some embodiments of the presently-disclosed subject matter involve targeting the reducing the expression of and/or activity of DENND5B to the intestines using a targeting moiety. In this regard, the DENND5B inhibitor can include a moiety for directing the inhibitor to the intestines.

In some embodiments of the presently-disclosed subject matter, the subject the subject is identified for treatment when the subject has diet-induced fatty liver disease; the subject has been identified as being at risk of developing diet-induced fatty liver disease; the subject has non-alcohol-induced fatty liver disease; the subject has been identified as being at risk of developing non-alcohol-induced fatty liver disease; the subject is obese as determined by BMI and/or abdominal circumference; the subject has type II diabetes; the subject has alcohol-induced fatty liver disease; and/or the subject has been identified as being at risk of developing alcohol-induced fatty liver disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1G. Knockout of Dennd5b in mice results in reduced plasma high density lipoprotein lipids and particle number. FIG. 1A: Genetic knockout of Dennd5b in mice using zinc-finger nuclease resulted in a 19 bp deletion (Cf. wild type SEQ ID NO: 2 with knock out SEQ ID NO: 3). Genotyping strategy utilizes PCR amplification of a DNA fragment containing the cut site and exploits loss of Bpil restriction site in mutant mice. FIG. 1B-1D: Total cholesterol, phospholipid, and triglyceride in plasma of wild type, heterozygous, and homozygous mice by gender. n=6-12/group on chow diet. FIGS. 1E and 1F: Size-exclusion chromatography analyses of plasma lipoproteins in wild type, heterozygous, and homozygous female mice. Each trace represents a pooled sample from n=3/group on chow diet. FIG. 1G: High density lipoprotein particle number was measured by nuclear magnetic resonance on a Vantera Lipoprotein Analyzer. n=3 pools of 5 female mice/group on chow diet. Statistical analyses were performed using 2-way ANOVA with Tukey post hoc tests (**p<0.01, ***p<0.001, ****p<0.0001). All values are mean±standard deviation.

FIGS. 2A-2M. Dennd5b^{-/-} mice have enlarged small intestine and reduced triglyceride absorption due to impaired chylomicron secretion by enterocytes. FIG. 2A: Picture of duodenal segments from overnight fasted wild type and Dennd5b^{-/-} mice. FIG. 2B: A 25 mm segment of duodenal small intestine, beginning 20 mm distal to pyloric sphincter, was cut longitudinally and laid flat for imaging and surface area calculation. Lumenal surface area was calculated as length×width of the segment in image pixels. n=6/group. ***p<0.0001 by unpaired t-test. FIG. 2C: Mice were given an oral gavage of vegetable oil (10 uL/g of body weight) and appearance of triglyceride in the plasma was measured at baseline, 2, and 4 hours post-gavage. n=10/group. *p<0.01 for wild type vs knockout using 2-way ANOVA with Sidak post hoc test. FIGS. 2D and 2E: Masson stained duodenal sections from wild type and Dennd5b^{-/-} mice 2 hours after oral oil-gavage. FIGS. 2F and 2G: Immunofluorescence stained duodenal sections 2 hours after oral oil-gavage. DAPI nuclear stain and anti-apoB. FIGS. 2H and 2I: Electron micrographs of duodenal tissue at one hour after oral oil-gavage. FIGS. 2J and 2K: Electron micrographs of duodenal tissue at two hours after oral oil-gavage. FIGS. 2L and 2M: Electron micrographs of duodenal tissue after an overnight fasting period. All values are mean±standard deviation. All mice used in these experiments were female.

FIGS. 3A-3M. Knockout of Dennd5b confers resistance to western diet induced weight gain, plasma and liver lipid increases, and atherosclerotic lesion development. FIG. 3A: Body weights of wild type and Dennd5b^{-/-} mice during 4-months on western diet. n=6/group. *p<0.0001 by 2-way ANOVA with Sidak post hoc test. FIG. 3B: NMR body composition analyses of wild type and Dennd5b^{-/-} mice after 4-months on western diet. n=6/group. **p<0.01 by 2-way ANOVA with Sidak post hoc test. FIG. 3C: Feces were collected from mice over a 3-day period and dried before measuring total mass. n=6-8/group. **p<0.01 by unpaired t-test. FIG. 3D-3F: Plasma total cholesterol, phospholipid, and triglyceride were measured during 4-months on western diet. *p<0.01 t-test with multiple comparisons correction by the Holm-Sidak method. FIG. 3G-3H: Size-exclusion chromatography analyses of plasma lipoproteins in wild type and homozygous mice after 4-months on western diet. Each trace represents a pooled sample from n=3/group. FIG. 3I: Quantification of lipid rich atherosclerotic lesions in the aortas of mice after 4-months on western diet. Aortas were harvested, stained with Sudan IV, and mounted for en face analyses. Plaque area was calculated as % of total aorta area cover by positive Sudan IV staining. *p<0.05 by unpaired t-test. FIG. 3J-3L: Lipids were extracted from liver after 4-months on western diet and total cholesterol, triglyceride, and free fatty acids were measured. n=6/group. *p<0.05 by unpaired t-test. FIG. 3M: VLDL production was measured by giving mice a retroorbital injection of tyloxapol and monitoring the appearance of triglyceride in the plasma over time. The rate of VLDL production was calculated from the slopes of the lines. All values are mean±standard deviation. All mice used in these experiments were female.

FIGS. 4A-4D. A common DENND5B genetic variant is associated with body mass index and abdominal circumference in humans. FIGS. 4A and 4B: An evaluation of body mass index (BMI) among wild type, heterozygous, and homozygous carriers of the p.(R52K) rs4930979 variant in females (A) and males (B). FIGS. 4C and 4D: An evaluation of abdominal circumference among wild type, heterozygous, and homozygous variant genotypes for the p.(R52K) variant in females (C) and males (D). Numbers at the base of each bar are the group mean. Statistical comparisons were performed by one-way ANOVA with post hoc correction by the Tukey method. *p<0.05, **p<0.01, n.s.=not significantly different. All values are mean±standard deviation.

FIG. 5A includes a photograph showing the liver of a Dennd5b^{+/+} mouse and FIG. 5B includes a photograph showing the liver of Dennd5b^{-/-} mice after being fed a Western diet (high-fat and high-cholesterol) for 12 weeks. Pale appearance of liver in Dennd5b^{+/+} mice on Western diet is consistent with significant lipid accumulation (FIG. 5A). In comparison, the liver in Dennd5b^{-/-} mice appears normal (FIG. 5B).

FIGS. 6A-6C include measurements of liver lipids, including cholesterol (FIG. 6A), triglycerides (FIG. 6B), and free fatty acids (FIG. 6C) for mice after being fed a Western diet (high-fat and high-cholesterol) for 12 weeks. Significantly reduced cholesterol and fatty acid levels were observed in Dennd5b^{-/-} mice.

FIGS. 7A and 7B include results showing the efficacy of using antisense oligonucleotides (ASOs) to inhibit Dennd5b gene expression (FIG. 7A) and protein levels (FIG. 7B) in mice.

FIG. 8 illustrates body weight as a function of time for the mice to whom Dennd5b ASOs were administered.

FIG. 9 includes measurements of CCL2 expression in the mice, as a marker of liver inflammation.

FIG. 10 depicts the reduction in plasma triglycerides in response to Dennd5b ASO treatment.

FIGS. 11A and 11B include results showing Dennd5b expression in response to various concentrations of Dennd5b ASOs.

### BRIEF DESCRIPTION OF THE SEQUENCE LISTING

SEQ ID NO: 1 is the nucleic acid sequence for Homo sapiens DENN domain containing 5B (DENND5B), which includes 9,506 nucleotides, wherein the open reading frame includes 3,824 nucleotides, starting after the first 304 nucleotides.

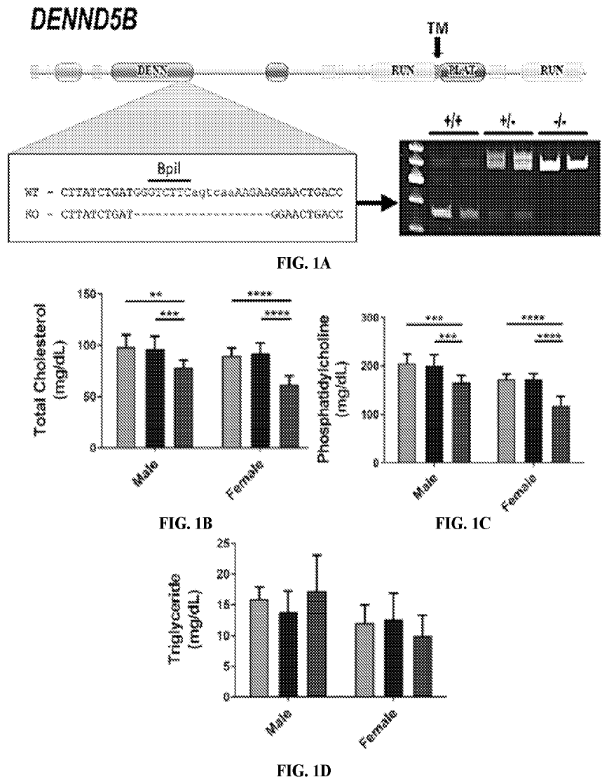

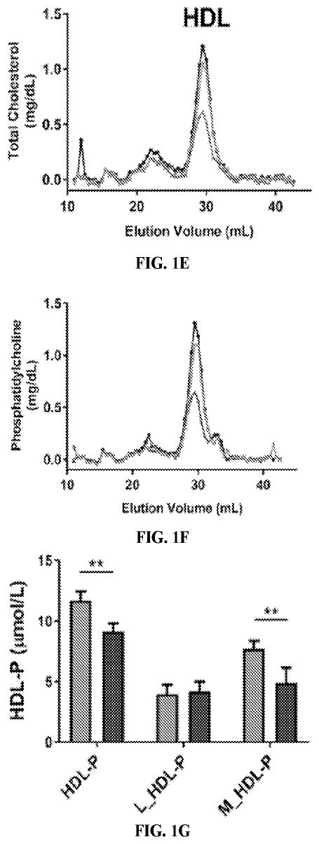

SEQ ID NO: 2 is the wild type (WT) sequence from FIG. 1A.

SEQ ID NO: 3 is the knockout (KO) sequence from FIG. 1A.

SEQ ID NOS: 4-15 are exemplary oligonucleotide sequences including consecutive nucleotides of SEQ ID NO: 1.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The present invention is based, in part, on the surprising data indicating a significant role in hepatic lipid metabolism for protein known as DENND5B (UniProt ID: Q6ZUT9). Prior this discovery as disclosed herein, this protein had no known function and virtually no presence in the scientific or medical literature.

The presently-disclosed subject matter includes methods of controlling absorption of and/or intracellular accumulation of triglycerides and lipids. In particular, certain embodiments of the presently-disclosed subject matter relate to methods of inhibiting DENND5B, thereby controlling absorption of and/or intracellular accumulation of triglycerides and lipids. Some embodiments of the presently disclosed subject matter include reducing lipid accumulation in the liver. Some embodiments of the presently disclosed subject matter include reducing lipid accumulation in the intestine. The presently-disclosed subject matter further includes methods of treating hepatic steatosis in a subject in need thereof or at risk therefore.

As disclosed herein, it is contemplated that specific targeting of DENND5B gene expression and/or activity in the liver can have a therapeutic effect for the common liver condition hepatic steatosis. Briefly, in a Dennd5b knockout mouse model, a remarkable resistance to diet induced lipid accumulation in the liver was observed. Human gene association studies also demonstrated that a common DENND5B gene variant is correlated with body mass index (BMI) and abdominal circumference. In humans, elevated BMI is a risk factor for fatty liver disease (hepatic steatosis).

The DENND5B gene and its protein product are highly expressed in the liver and represent a unique target for the treatment of hepatic lipid accumulation. The data disclosed herein suggest that reducing the amount of this protein in the liver may provide protection.

Some embodiments of the presently-disclosed subject matter include a method of treating hepatic steatosis, which involves identifying a subject having or at risk of developing hepatic steatosis, and reducing the expression of and/or activity of DENND5B in the liver of the subject.

Some embodiments of the presently-disclosed subject matter include a method of reducing lipid accumulation in the intestines. The presently-disclosed subject matter further includes methods of controlling intestinal triglyceride absorption. Some embodiments of the presently-disclosed subject matter include a method of reducing lipid accumulation in the intestines, which involves reducing the expression of and/or activity of DENND5B in intestines of a subject.

Some embodiments of the presently-disclosed subject matter, the method involves administering to the subject a DENND5B inhibitor. In some embodiments, the DENND5B inhibitor reduces the expression of DENND5B in the liver. In some embodiments, the DENND5B inhibitor reduces the activity of DENND5B in the liver. In some embodiments, the DENND5B inhibitor comprises antisense oligonucleotide (ASO), CRISPR interference (CRISPRᵢ), miRNA, siRNA, locked nucleic acid (LNA) nucleotides, or a combination thereof.

As will be recognized by one of ordinary skill in the art, inhibitors that operate using nucleotide interference can make use of a nucleotide sequence or a guide antisense strand that is designed in in view of the sequence of the target gene of interest. If a specific target gene and its sequence are identified, interference molecules, such as ASOs can be designed. ASOs can be prepared using methods known to those of ordinary skill in the art, such as those described, for example, in Aartsma-Rus (2009), Integrated DNA Technologies (2011), and Yu (2016). In this regard, custom ASOs can be obtained commercially from various companies by providing the company with information including the sequence of a specific target gene of interest. In the presently-disclosed subject matter, the target gene of interest is DENND5B, the nucleotide sequence of which is provided in SEQ ID NO: 1.

In some embodiments, the DENND5B inhibitor comprises a guide antisense strand consisting of about 10, 11, 12, 13, 14, 15, 16, 17, 18, 19, 20, 21, 22, 23, 24, 25, 26, 27, 28, 29, or 30 nucleotides. In some embodiments, the guide antisense strand consisting of about 10, 11, 12, 13, 14, 15, 16, 17, 18, 19, 20, 21, 22, 23, 24, 25, 26, 27, 28, 29, or 30 nucleotides that are complementary to consecutive nucleotides of SEQ ID NO: 1. In some embodiments, the guide antisense strand consisting of about 10, 11, 12, 13, 14, 15, 16, 17, 18, 19, 20, 21, 22, 23, 24, 25, 26, 27, 28, 29, or 30 consecutive nucleotides of SEQ ID NO: 1. In some embodiments, the guide antisense strand consisting of about 10, 11, 12, 13, 14, 15, 16, 17, 18, 19, 20, 21, 22, 23, 24, 25, 26, 27, 28, 29, or 30 nucleotides that are complementary to consecutive nucleotides of residues 1-2216 of SEQ ID NO: 1. In some embodiments, the guide antisense strand consisting of about 10, 11, 12, 13, 14, 15, 16, 17, 18, 19, 20, 21, 22, 23, 24, 25, 26, 27, 28, 29, or 30 consecutive nucleotides of residues 1-2216 of SEQ ID NO: 1.

In some embodiments, the guide antisense strand comprises at least eight (8) nucleotides that are guanine (G) or cytosine (C). In some embodiments, the guide antisense strand comprises at least eight (7) nucleotides that are guanine (G) or cytosine (C). In some embodiments, the guide antisense strand comprises nucleotides in which at least about 25, 30, 35, 40, 45, 50, 55, 60, 65, 70, 75, 80, 85, 90, 95, or 100% of the nucleotides are guanine (G) or cytosine (C).

In some embodiments, the guide antisense strand comprises nucleotides of a predicted serine/arginine-rich (SR) protein binding site, or a complement thereof.

In some embodiments, the SR protein binding site is an SR splicing factor 1 (SRSF1) binding site. In some embodiments, the SR binding site comprises seven (7) consecutive nucleotides of SEQ ID NO: 1, beginning with the nucleotide at residue 6, 19, 27, 50, 53, 71, 82, 86, 87, 94, 108, 120, 134, 141, 157, 178, 190, 199, 206, 213, 217, 224, 225, 228, 230, 233, 249, 264, 268, 271, 274, 277, 297, 298, 307, 324, 329, 351, 358, 369, 399, 403, 417, 429, 457, 486, 495, 571, 575, 579, 635, 653, 691, 702, 726, 736, 856, 889, 891, 952, 954, 966, 1028, 1073, 1155, 1201, 1203, 1217, 1220, 1233, 1246, 1337, 1389, 1443, 1455, 1494, 1524, 1531, 1547, 1562, 1646, 1704, 1785, 1812, 1836, 1838, 1871, 1878, 1898, 1903, 1947, 1952, 1995, 1997, 2034, 2036, 2074, 2123, 2163, 2186, or 2201 of SEQ ID NO: 1.

In some embodiments, the SR protein binding site is an SR splicing factor 2 (SRSF2) binding site. In some embodiments, the SR binding site comprises eight (8) consecutive nucleotides of SEQ ID NO: 1, beginning with the nucleotide at residue 10, 17, 36, 45, 54, 76, 91, 92, 114, 121, 127, 152, 168, 190, 219, 243, 281, 322, 329, 335, 341, 352, 356, 391, 400, 408, 412, 443, 450, 485, 511, 570, 580, 590, 629, 819, 833, 910, 943, 960, 1032, 1077, 1082, 1084, 1131, 1140, 1254, 1299, 1321, 1328, 1343, 1344, 1407, 1414, 1438, 1459, 1498, 1516, 1588, 1641, 1651, 1718, 1728, 1787, 1816, 1843, 1889, 1921, 2002, 2065, 2080, 2098, 2110, 2184, or 2197 of SEQ ID NO: 1.

In some embodiments, the SR protein binding site is an SR splicing factor 5 (SRSF5) binding site. In some embodiments, the SR binding site comprises seven (7) consecutive nucleotides of SEQ ID NO: 1, beginning with the nucleotide at residue 13, 49, 59, 81, 163, 221, 283, 416, 442, 456, 487, 551, 552, 565, 574, 601, 615, 639, 672, 690, 709, 733, 773, 801, 829, 853, 888, 937, 953, 962, 974, 979, 991, 1023, 1034, 1044, 1072, 1108, 1133, 1195, 1198, 1202, 1224, 1242, 1245, 1264 1301, 1313, 1352, 1388, 1409, 1420, 1454, 1493, 1523, 1643, 1657, 1662, 1673, 1692, 1722, 1730, 1744, 1754, 1789, 1811, 1833, 1835, 1872, 1929, 1948, 1977, 1994, 2033, 2035, 2071, 2073, 2104, 2153, 2200, or 2202 of SEQ ID NO: 1.

In some embodiments, the SR protein binding site is an SR splicing factor 6 (SRSF6) binding site. In some embodiments, the SR binding site comprises six (6) consecutive nucleotides of SEQ ID NO: 1, beginning with the nucleotide at residue 4, 27, 33, 69, 118, 124, 133, 155, 176, 188, 194, 215, 222, 249, 290, 295, 319, 359, 362, 374, 386, 397, 529, 545, 549, 691, 699, 734, 742, 748, 751, 766, 906, 949, 954, 957, 986, 1155, 1228, 1238, 1297, 1411, 1604, 1625, 1633, 1674, 1689, 1699, 1705, 1726, 1761, 1803, 1836, 1846, 1917, 1982, 1995, 2029, 2058, 2097, 2121, 2131, 2133, 2144, or 2161 of SEQ ID NO: 1.

In some embodiments, the DENND5B inhibitor comprises a guide antisense strand comprising a sequence, or a complement thereof, as set forth in Table 1. The sequences set forth in Table 1 include consecutive nucleotides of SEQ ID NO: 1, including sequences beginning with the nucleotide at residue 192, 663, 751, 896, 944, 1014, 1061, 1145, 1167, 1664, 1859, or 2090 of SEQ ID NO: 1.

TABLE 1

| | SEQ |
|---|---|
| Sequence | ID NO: |
| CCAGTGTCGCCCTAGGGCGGGCGCACAGAC | 4 |
| ATGAAGAAGTTACAAGTAAGCAAATCTGCA | 5 |
| TTCATCTTCCTGCAGTATGGACTCATTGGC | 6 |
| TTACCATTCATGCAGGCCTGCAAGAAATTC | 7 |
| GCTGTTACCTCACAGCAGCCACCACCCTTG | 8 |
| CCCTTCCACCTCCAGGGAGGTCACTGAAAT | 9 |
| CCTGTCATCTGCCAGAGGCCTGGGCCCAGT | 10 |
| GAGAACCTGGTGCAGGTGTTTACCTGTGTT | 11 |
| CCTGTGTTCTTTTAGAGATGCAAATCCTTC | 12 |
| ATAGCCCGCTTGCAGGCTCTGGCCAAGCGT | 13 |
| GCATTTGTCATTCAGACTGCCCAGGACATG | 14 |
| ATAAGGCTGTATAATGTAAGGGCACCCACC | 15 |

In some embodiments, the guide antisense strand has a Tₘ of greater than about 45, 46, 47, 48, 49, 50, 51, 52, 53, 54, 55, 56, 57, 58, 59, 60, 61, 62, 63, 64, or 65° C. In some embodiments, the guide antisense strand has a Tₘ of greater than about 48° C.

In some embodiments of the presently-disclosed subject matter, the reduction in expression of DENND5B is achieved using a nucleotide molecule targeting a protein coding region of DENND5B gene. In some embodiments of the presently-disclosed subject matter, the reduction in expression of DENND5B is achieved using a nucleotide molecule targeting a non-coding region of DENND5B gene. In some embodiments, the nucleotide molecule targets promoter of DENND5B gene. In some embodiments, the nucleotide molecule targets 3′ untranscribed regions (UTRs) of DENND5B gene.

Some embodiments of the presently-disclosed subject matter involve targeting the reducing the expression of and/or activity of DENND5B to the liver using a targeting moiety. In this regard, the DENND5B inhibitor can include a moiety for directing the inhibitor to the liver.

Some embodiments of the presently-disclosed subject matter involve targeting the reducing the expression of and/or activity of DENND5B to the intestines using a targeting moiety. In this regard, the DENND5B inhibitor can include a moiety for directing the inhibitor to the intestines.

In some embodiments of the presently-disclosed subject matter, the subject has or is at risk of developing diet-induced fatty liver disease. In some embodiments, the subject has or is at risk of developing non-alcohol-induced fatty liver disease. In some embodiments, the subject is identified as obese as determined by BMI and/or abdominal circumference. In some embodiments, the subject is identified has having type II diabetes. In some embodiments, the subject has or is at risk of developing alcohol-induced fatty liver disease.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9): 1726-1732).

Although any methods, devices, and materials similar or equivalent to those described herein can be used in the practice or testing of the presently-disclosed subject matter, representative methods, devices, and materials are described herein.

The terms “treatment” or “treating” refer to the medical management of a subject with the intent to cure, ameliorate, reduce, or prevent hepatic steatosis. As will be recognized by one of ordinary skill in the art, the term “cure” does not refer to the ability to completely remove all excess lipid accumulation in a target area. For example, in some embodiments, a cure can refer to a decrease at a level of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. Similarly, as will be recognized by one of ordinary skill in the art, the term “prevent” does not refer to an ability to completely remove any and all lipid accumulation.

Likewise, as will be recognized by one of ordinary skill in the art, the term “inhibiting” or “inhibition” does not refer to the ability to completely inactivate all target biological activity in all cases. Rather, the skilled artisan will understand that the term “inhibiting” refers to decreasing biological activity of a target, such as a DENND5B, such as can occur, for example, when a nucleotide limits the expression of the target gene, when a ligand binding site of the target protein is blocked, or when a non-native complex with the target is formed. Such decrease in biological activity can be determined relative to a control, wherein an inhibitor is not administered and/or placed in contact with the target. For example, in some embodiments, a decrease in activity relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. The term “inhibitor” refers to a compound of composition that reduces the expression of and/or decreases the biological activity of a target, such as a DENND5B.

The terms “subject” or “subject in need thereof” refer to a target of administration, which optionally displays symptoms related to a particular disease, pathological condition, disorder, or the like. The subject of the herein disclosed methods can be a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some embodiments of the methods disclosed herein, the subject and/or patient does not have cancer.

The term “administering” refers to any method of providing a therapeutic composition to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

Relevant information regarding DENND5B (UniProt ID: Q6ZUT9) can be found at the following locations, and reference thereto is made to the information accessible as of the filing date of this application: www.uniprot.org/uniprot/Q6ZUT9; www.genecards.org/cgi-bin/carddisp.pl?gene=DENND5B; horfdb.dfci.harvard.edu/hv7/index.php?page=getresults&by=detail&qury=71222; and www.ncbi.nlm.nih.gov/gene?Db=gene&Cmd=DetailsSearch&Term=160518

The term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

Example 1: Generation of Dennd5b^{-/-} mice. A custom zinc finger nuclease (ZFN) was generated to target an exonic sequence within the first third of the Dennd5b gene. The ZFN RNA (20-40 ng/uL) was administered to fertilized B6CBAF1 mouse embryos by pronuclear injection and embryos were implanted into females. Resulting pups were genotyped, using PCR and Sanger sequencing. A Dennd5b^{-/-} line with a biallelic 19 bp deletion was established. This mutation predicts a frameshift and early termination due to the introduction of a stop codon. Wild type mice (WT) on the same background were used as controls for all experiments. Mice were fed standard chow (Envigo 7017) or western diet (WD) with 42% calories from fat (Harlan TD.88137). Western diet studies were initiated in mice beginning at 2 months of age. For all other experiments mice used were between 2-4 months of age and controls were age matched (within one week).

Example 2: Blood collection and plasma lipid analyses. Mouse blood was collected by retro-orbital bleed, using heparinized capillary tubes and added to tubes containing EDTA. Plasma was obtained by centrifugation at 3,000 rpm for 20 min. at 4° C. Plasma lipids were measured using colorimetric enzymatic assays (Wako Diagnostics). Size-exclusion chromatography separations of plasma lipoproteins were performed on an Akta Pure instrument equipped with two Superose 6 (GE Healthcare) columns arranged in series. Plasma (100 μL) was run over columns at a flow rate of 0.5 mL/min in Tris buffer (10 mM Tris, 150 mM NaCl, 0.5 mM EDTA, 0.01% sodium azide) and 0.5 mL fractions collected.

Example 3: Lipid absorption studies. Mice were fasted overnight (≥16 hours) before lipid absorption experiments. Vegetable oil (10 uL/g body weight) was administered by oral gavage, using a blunt ball-tipped syringe. Plasma was collected as described above, at time points indicated, and triglyceride measured by enzymatic assay.

Example 4: VLDL secretion assay. Mice were fasted overnight (16 hours) and baseline blood collected prior to retroorbital injection (6 μL/g body weight) of Tyloxapol in saline (5% wt:vol). Blood was collected from mice by retroorbital bleed from eye opposite to injection site at 1, 2, and 4 hours post-injection. Plasma was analyzed for triglyceride content by enzymatic assay.

Example 5: Hepatic lipid analysis. Liver tissue harvested from PBS perfused mice was snap frozen with liquid nitrogen and stored at −80° C. until lipid extraction. A portion of liver (80-100 mg) from the lateral left lobe was used for lipid extraction. Tissue was added to 1 mL 0.9% NaCl, minced and lipids collected by chloroform methanol extraction. Chloroform layer containing lipid was dried under nitrogen gas. Dry lipid was solubilized in hexane isopropanol (3:2, v:v) and a small volume used for measurement of cholesterol, triglyceride, and free fatty acids by enzymatic assay. Measurements were adjusted to the starting liver tissue mass.

Example 6: Fecal lipid analysis. Feces were collected from mice over a 3-day period and dried before measuring total mass. About 250 mg of dry feces was pulverized, using a mortar and pestle, and total lipids were isolated by chloroform methanol (2:1, v:v) extraction. Chloroform layer containing lipid was dried under nitrogen gas and weighed to determine total lipid mass. Dry lipid was solubilized in hexane isopropanol (3:2, v:v) and a small volume used for measurement of cholesterol, triglyceride, and free fatty acids by enzymatic assay (All from Wako Diagnostics). Measurements were adjusted to the starting fecal mass.

Example 7: Electron microscopy of small intestine. Mice were sacrificed by cervical dislocation and a 1 mm section of duodenal small intestine tissue was quickly harvested beginning 1 mm distal to the pyloric sphincter of the stomach. Tissue was immediately placed in Karnovsky's fixative and chopped into cubes 1 mm³ or less, post-fixed in osmium tetroxide, dehydrated and embedded in Epon for sectioning. Sections on uncoated grids were imaged on a JEOL JEM 1200EXII transmission electron microscope.

Example 8: Aortic atherosclerosis lesion area quantification. Mouse aortas, from base of aortic arch to the femoral branch, were harvested and fixed in fresh 4% paraformaldehyde for 5 minutes before staining with Sudan IV (0.5%, wt:vol) for 15 minutes. After staining, aortas were destained for 15 minutes with 80% ethanol and stored in water. Scissors were used to open the aortas for en face analyses. Color images were taken along the full length of the aorta and imaging software was used to quantify stained lesions. Atherosclerotic lesion area was calculated as (stain positive area/total aortic surface area)*100.

Example 10: DENND5B genotype association studies in Humans. The ClinSeq® exome sequencing cohort¹¹ comprising 621 participants with reliable sequence data for the DENND5B Arginine 52 codon (variant rs4930979), were used for analysis. Body mass and blood lipid parameters were evaluated across homozygous reference, heterozygous, and homozygous variant genotypes for the c.155G>A; p.(R52K) variant by ANOVA with Tukey correction for multiple comparisons.

The Mayo Vascular Disease Biorepository (VDB) was used for replication analyses¹². Genotyping of 9,274 VDB participants was performed, using genome-wide SNP arrays on Illumina platforms. Genotype data were imputed using Michigan Imputation Server¹³ based on a human reference consortium (HRC, r1.1) panel¹⁴. Genotype association tests for lipid levels and BMI were based on linear regression analyses assuming an additive effect with adjustment for age, sex and the first two principal components.

Example 11: Dennd5b^{-/-} mice have low plasma HDL. A Dennd5b^{-/-} mouse line was generated, using a zinc finger nuclease. With reference to FIG. 1A, this approach resulted in a 19 bp deletion (Cf. SEQ ID NO: 2 with SEQ ID NO: 3), predicting a frameshift mutation and early termination signal. Mice were fertile with outwardly normal morphology.

With reference to FIGS. 1B-1D, measurement of plasma lipids in Dennd5b^{-/-} mice revealed a significant reduction in plasma total cholesterol (TC) and phospholipids (PL) but not triglyceride (TG). Reduced lipids were only present in homozygous mice, not in heterozygous, and the effect was greater in females than in males (−30% vs. —20%). Because female Dennd5b^{-/-} mice demonstrated a more prominent plasma lipid phenotype, female mice were used for subsequent experiments.

Referring to FIGS. 1E and 1F, size-exclusion chromatography of mouse plasma demonstrated that the reduction in lipids was attributed entirely to a reduction of HDL-sized lipoproteins. Nuclear magnetic resonance analyses of lipoprotein particle concentrations indicated a 22% reduction in total HDL particle number (HDL-P), which was entirely due to a lower concentration of medium sized HDL particles, as reflected in FIG. 1G. Large HDL particle numbers were not affected.

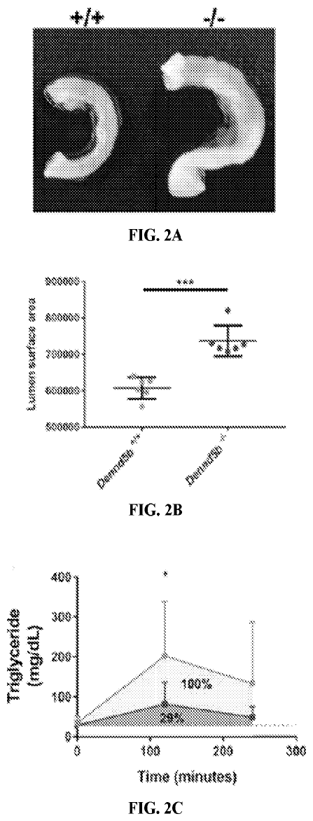

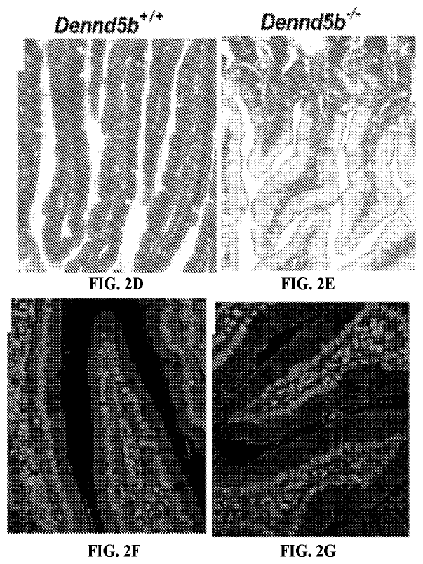

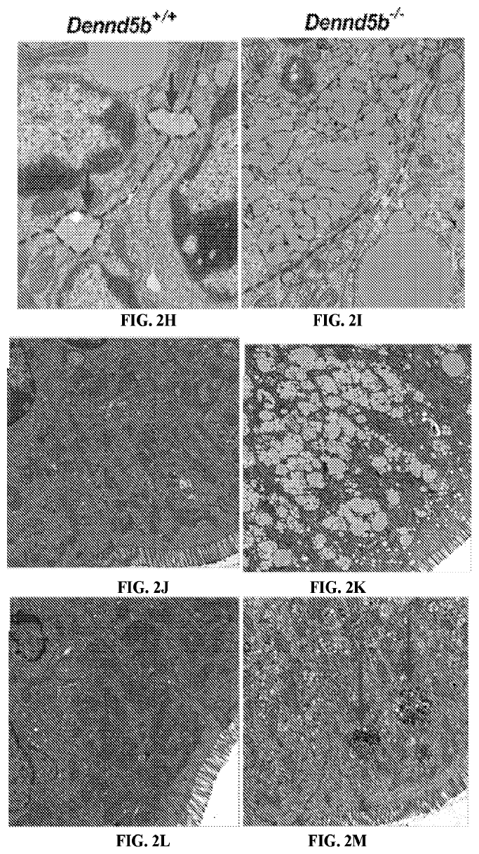

Example 12: Impaired intestinal triglyceride absorption in Dennd5b^{-/-} mice. Gross inspection of all internal organs of the Dennd5b^{-/-} mice was normal, except for the small intestine. Dennd5b^{-/-} mice on chow diet had a distended small intestine, as depicted in FIG. 2A, quantified by a 20% increase in luminal surface area, as shown in FIG. 2B. The small intestine had a whitish color consistent with possible fat accumulation, even though the mice had been fasting overnight. To evaluate transit of dietary triglyceride to the plasma, oil-gavage studies were performed in four-month-old fasting mice. These studies showed significantly lower plasma triglyceride (see FIG. 2C) and free fatty acids at 2 hours post-gavage in Dennd5b^{-/-} mice compared to wild type controls. With reference to FIGS. 2D and 2E, microscopic analyses of duodenal sections showed massive lipid accumulation in Dennd5b^{-/-} enterocytes. Additionally, with reference to FIGS. 2F and 2G, immunofluorescence confocal microscopy of duodenal sections indicated that secretion of apoB into the lacteal was dramatically lower in Dennd5b^{-/-} small intestine. Taken together, these data suggest a significant impairment of intestinal TG absorption in Dennd5b^{-/-} mice.

Example 13: Dennd5b is involved in post-Golgi chylomicron secretion by enterocytes. To gain insight into the mechanism of the involvement of Dennd5b in intestinal TG absorption, transmission electron microscopy was used to examine duodenal enterocytes in wild type (WT) and Dennd5b^{-/-} mice. Imaging of tissue at various time points after oral oil gavage allowed for visualization of the sequential steps of chylomicron secretion.

At 1 hour post-gavage, fusion events between chylomicron secretory vesicles and the plasma membrane result in secretion of chylomicrons by WT enterocytes, as shown in FIG. 2H. With reference to FIG. 2I, in Dennd5b^{-/-} mice, these fusion events are rare and very few extracellular chylomicrons are observed. At 2 hours, WT enterocytes have cleared the majority of lipid, as shown in FIG. 2J; however, as shown in FIG. 2K there is significant accumulation of fat in Dennd5b^{-/-} enterocytes in the form of vesicle-bound chylomicrons and some lipid droplets. These images revealed similar lipid droplet formation, pre-chylomicron formation in the ER lumen, pre-chylomicron transport vesicles (PCTV's), chylomicron maturation in the Golgi and budding of chylomicron secretory vesicles (CSVs) from the Golgi in wild type and Dennd5b^{-/-} mice. However, in contrast to wild type mice, Dennd5b^{-/-} enterocytes exhibited massive accumulation of CSV's, which appeared to be unable to fuse with the basolateral plasma membrane. Whereas WT mice secreted chylomicrons into the intercellular space and the lamina propria by 1 hour after gavage, chylomicron secretion by Dennd5b^{-/-} enterocytes was rarely observed.

Together, these findings support a role for Dennd5b in post-Golgi transport of chylomicron secretory vesicles. With reference to FIGS. 2L and 2M, enteric lipid accumulation was even observed in Dennd5b^{-/-} mice that did not receive an oil gavage and had undergone an overnight period of fasting. In Dennd5b^{-/-} enterocytes, electron dense structures similar to intracellular digestive vesicles were observed (see FIG. 2M, arrow), which may represent an alternative pathway for the removal of excess intracellular TG by autophagy.

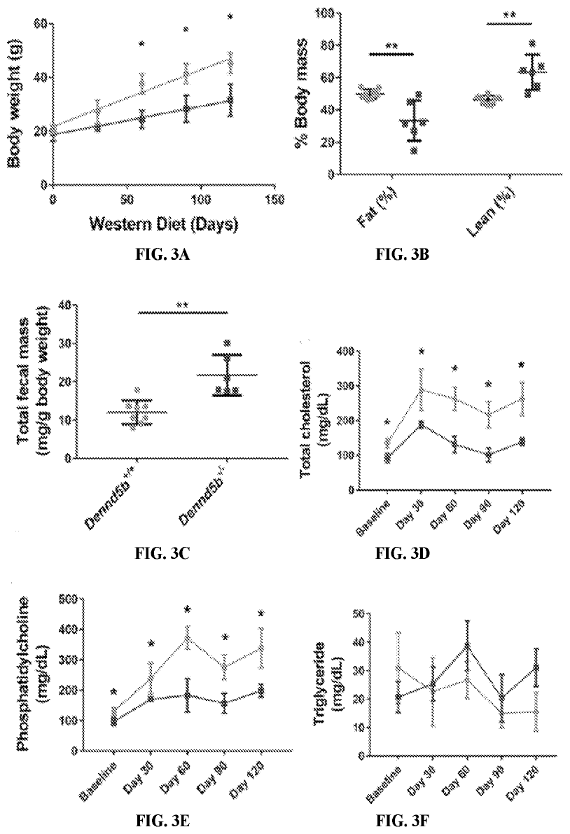

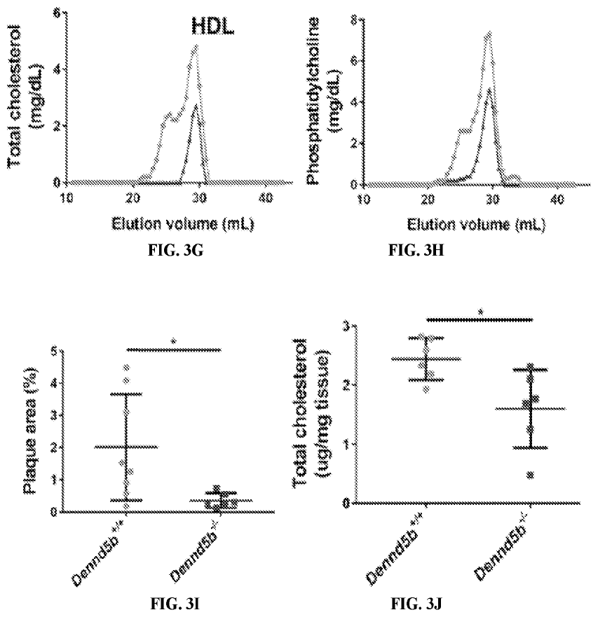

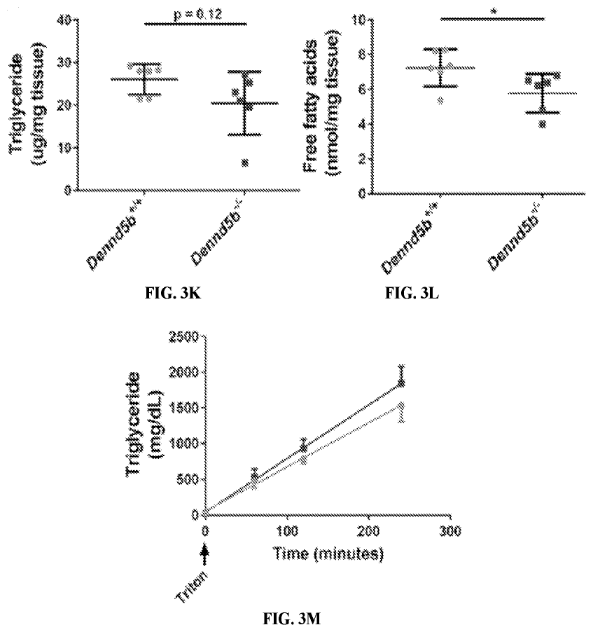

Example 14: Dennd5b^{-/-} mice are resistant to western diet induced weight gain and changes in plasma lipids. Dennd5b^{-/-} mice and age-matched wild type controls were placed on western diet (WD, 42% calories from fat and 0.2% cholesterol), beginning at 2 months of age. The rate of body weight gain in Dennd5b^{-/-} mice was about half the rate of WT mice, as shown in FIG. 3A. After 4 months on WD, the body weight of Dennd5b^{-/-} mice was 30% lower than WT mice. The difference in body weight was explained by a shift in body mass composition, with Dennd5b^{-/-} mice having lower fat mass and greater lean mass, as reflected in FIG. 3B.

On standard chow diet, WT and knockout mice maintain similar body weight. On WD, fecal lipids were measured. Although total fecal mass was increased in Dennd5b^{-/-} mice, as shown in FIG. 3C, significant increases in total lipid content were not detected, although there was a trend toward increased total lipids in Dennd5b^{-/-} feces. Measurement of specific lipid components revealed that fecal total cholesterol and triglyceride content were not different but fecal free fatty acid content was significantly increased in the knockout mice.

In plasma, TC and PL increased compared to baseline values in WT mice and there was no change in triglyceride. However, with reference to FIGS. 3D and 3F, Dennd5b^{-/-} mice were resistant to WD-induced plasma lipid increases. With reference to FIGS. 3G and 3H, while on WD, WT mice maintained a higher amount of HDL and accumulated a larger-sized population of cholesterol-rich lipoprotein, likely small LDL, that was absent in Dennd5b^{-/-} mice. The favorable plasma lipid and body composition profile in Dennd5b^{-/-} mice suggested a possible protective effect against diet-induced atherosclerosis. With reference to FIG. 3I, analyses of atherosclerotic lesion area in mice after 4 months on WD showed significantly lower plaque burden in Dennd5b^{-/-} mice compared to wild type controls.

Example 15: Dennd5b^{-/-} mice compensate for lack of dietary lipid by increasing hepatic VLDL production. To evaluate the effect of Dennd5b knockout on hepatic lipid metabolism in mice on WD, lipid was extracted from liver tissue. With reference to FIGS. 3J-3L, TC and FFA were significantly reduced in Dennd5b^{-/-} mouse liver and there was a trend toward decreased TG. The VLDL production by the liver was evaluated by measuring TG appearance in plasma of fasting mice after intravenous tyloxapol injection. As shown in FIG. 3 M, Dennd5b^{-/-} mice had a 21% increase in rate of VLDL associated TG accumulation compared to WT (slope=7.5 vs 6.18, p=0.0098).

Example 16: Dennd5b expression correlates with liver mass and lipid content. The Hybrid Mouse Diversity Panel (HMDP) is a collection of approximately 100 well-characterized inbred strains of mice that can be used to analyze the genetic and environmental factors underlying complex traits. Bennett 2015, Lusis 2016). Data extracted from the HMDP demonstrates that expression level of Dennd5b correlates with liver traits. With reference to Table 2, of the 10 strongest relationships uncovered by this screen, eight relate to the liver, including total liver mass and average fat mass.

TABLE 2
Mouse hepatic Dennd5b expression correlates
with traits (top 10, by P-value)

| Gene | Trait | Trait category | P-value |
|---|---|---|---|
| Dennd5b | Liver/body weight | tissue weights | 2.28E−16 |
| Dennd5b | Liver NMR | tissue weights | 8.41E−13 |
| Dennd5b | average lean mass (liver) | liver NMR | 1.31E−12 |
| Dennd5b | average total mass (liver) | liver NMR | 4.11E−12 |
| Dennd5b | Liver Total | tissue weights | 4.90E−12 |
| Dennd5b | Liver Hist | tissue weights | 8.49E−11 |
| Dennd5b | Fibrosed area | liver fibrosis | 5.52E−09 |
| Dennd5b | Spleen | tissue weights | 2.51E−07 |
| Dennd5b | average fat mass (liver) | liver NMR | 4.82E−07 |
| Dennd5b | free fatty acids | blood lipids | 9.00E−06 |

Italicized rows indicate correlations between Dennd5b and liver traits.

Blood level of free fatty acids was also correlated. This measure is often closely related to hepatic lipid metabolism and liver health. These data demonstrate that expression level of Dennd5b is related to liver mass and lipid content in diverse population of mice.

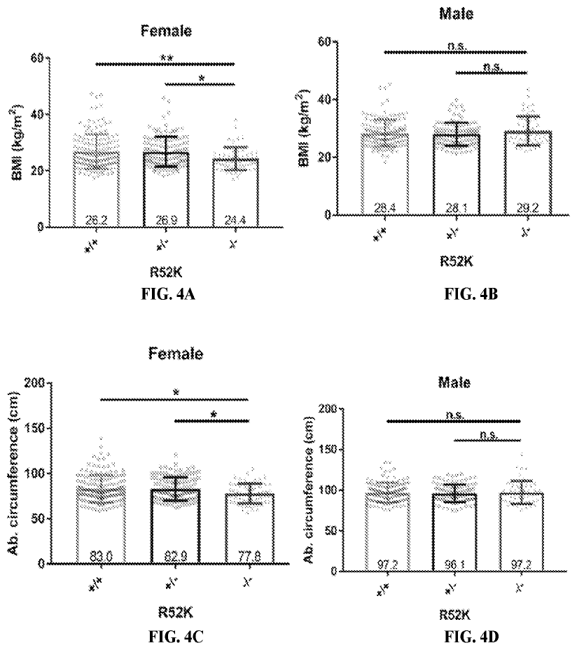

Example 17: A common DENND5B variant is associated with body mass index in humans. The human homologue (DENND5B) is 95% identical to the mouse protein. To determine if DENND5B plays a similar role in human physiology, the influence of a common gene variant, p. (R52K) (rs4930979), on body weight and plasma lipids was examined in the ClinSeq® exome sequencing study (n=621). With reference to FIGS. 4A and 4C, females homozygous for the variant allele had significantly lower BMI (24.4 vs. 26.9, p=0.008, n=329) and abdominal circumference (77.8 vs. 83.0, p=0.03, n=330), when compared to homozygous reference. The effect on BMI was due to increased body weight, no difference in height was observed. The p.(R52K) variant did not influence these measures in male participants, as reflected in FIGS. 4B and 4D, and did not correlate with plasma lipids for either gender in this cohort.

In an independent, larger cohort (Mayo Vascular Disease Biorepository, VDB), p.(R52K) (MAF=0.3984) was associated with BMI (β=−0.179, P=0.041, n=8,303). In this dataset, another DENND5B variant p.(H487N) (rs1056320, MAF=0.1072) was associated with LDL-C (β=−2.03, P=0.0064, n=8,571); however, this variant was not significantly associated with BMI (β=−0.217, P=0.11, n=8,303). One explanation for the differing metabolic presentations associated with these variants is that they have distinct influences on protein function due to their location in the protein. For example, DENND5B could perform more than one function with influence on metabolism.

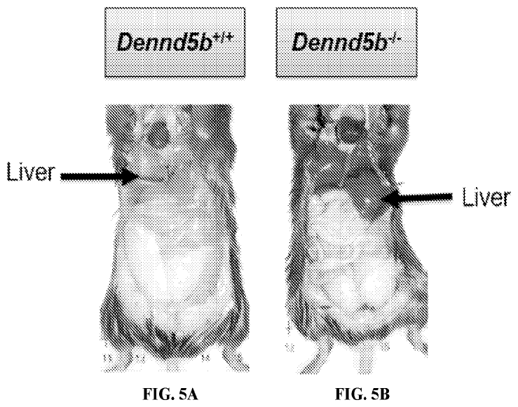

Example 18: Dennd5b−/− mice are resistant to diet-induced hepatic lipid accumulation. After 12 weeks of being feed a Western diet, the livers of Dennd5b^{+/+} and Dennd5b^{-/-} mice were observed. With reference to FIG. 5A, pale appearance of liver in Dennd5b^{+/+} mice on Western diet is consistent with significant lipid accumulation. In comparison, with reference to FIG. 5B, the liver in Dennd5b^{-/-} mice appears normal. The Dennd5b^{-/-} mice are resistant to diet-induced hepatic lipid accumulation.

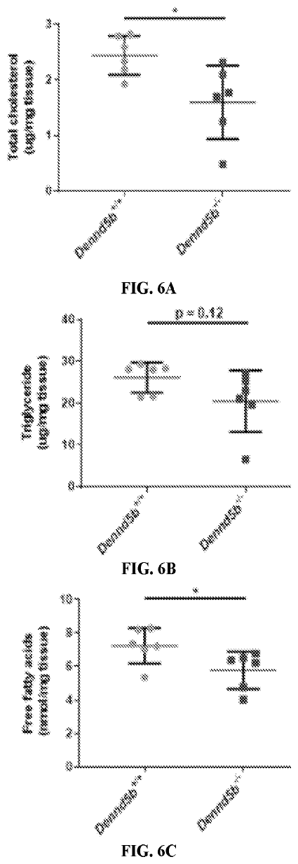

Example 19: Dennd5b−/− mice have reduced liver lipid levels, including significantly reduced cholesterol and fatty acid levels. After being fed a Western diet, liver lipids were measured in Dennd5b^{-/-} and Dennd5b^{+/+} mice. With reference to FIG. 6A, cholesterol was significantly reduced in Dennd5b^{-/-} mice. With reference to FIG. 6B, triglyceride levels were reduced in Dennd5b^{-/-} mice. With reference to FIG. 6C, fatty acid levels were significantly reduced in Dennd5b^{-/-} mice.

Example 20: Suppression of hepatic Dennd5b expression in mice. Suppression of Dennd5b expression in mice was conducted using antisense oligonucleotide (ASO). ASOs can be used to modify expression of specific target genes having a known sequence. If a specific target gene and its sequence are identified, ASOs can be prepared using methods known to those of ordinary skill in the art. (See, e.g., Aartsma-Rus (2009), Integrated DNA Technologies (2011), Yu (2016)). In this regard, custom ASOs can be obtained commercially from various companies by providing the company with information including the sequence of a specific target gene of interest. For studies described in this Example, information identifying Dennd5b as a desired target of interest was provided to Ionis Pharmaceuticals, Inc., who provided the present inventors with eight (8) exemplary ASOs for testing.

C57BL-6J mice were obtained. ASOs were injected subcutaneously at a dose of about 75 mg/kg. Each one of the eight ASOs was injected into multiple mice (at least n=4). A total of 40 mice were used. Injections occurred on day 1, day 3, day 7, and day 14. On day 21, samples were harvested to assess Dennd5b RNA and protein levels in liver, blood chemistry, liver enzymes, and plasma lipids.

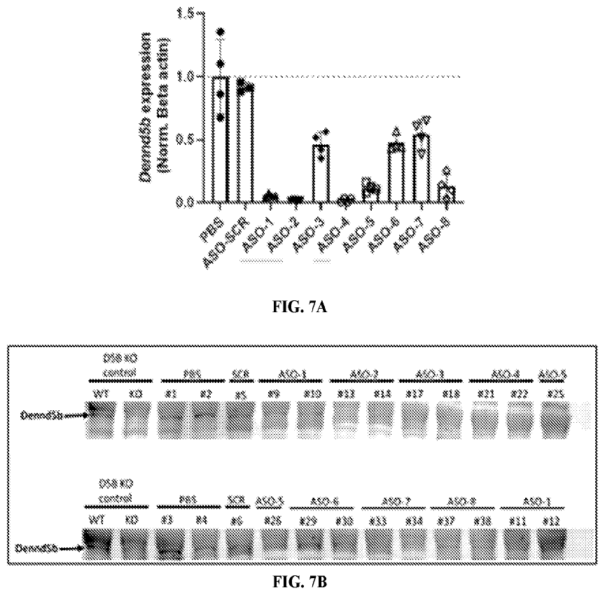

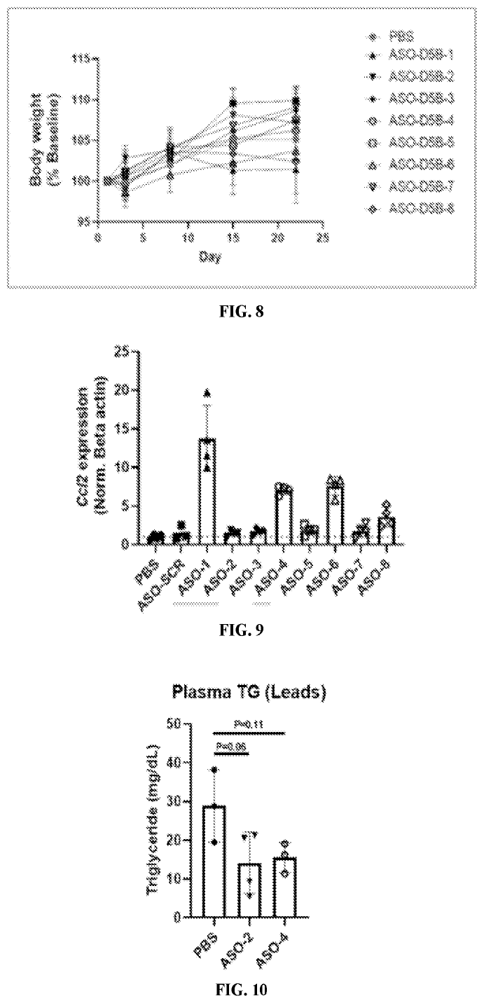

As reflected in FIGS. 7A and 7B, some embodiments resulted in greater than about a 20% knockdown efficiency; however, all of the ASOs were effective in inhibiting Dennd5b gene expression and protein levels. With reference to FIG. 7A, all of the ASOs resulted in an inhibition of Dennd5b gene expression. Also, as shown in FIG. 7B, all of the ASOs resulted in decreased Dennd5b protein levels. Meanwhile, with reference to FIG. 8, none of the exemplary ASOs had a negative impact on body weight.

Two of the exemplary ASOs (ASO-2 and ASO-4) were identified for further study, in view of their favorable inhibition of Dennd5b (FIG. 7A), as well as having a favorable liver inflammation profile as assessed by expression of a marker of liver inflammation, CCL2 (FIG. 9).

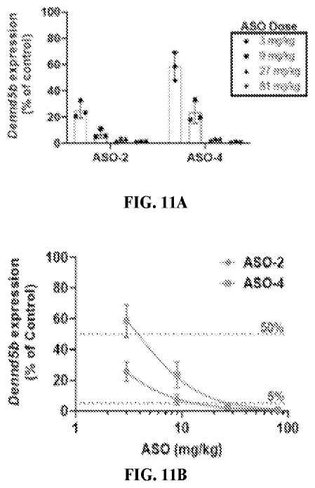

The effect of the two exemplary ASOs (ASO-2 and ASO-4) on plasma lipids under basal conditions was assessed. As reflected in FIG. 10, there was a reduction in plasma triglycerides in response to ASO treatment, providing an indication that the ASO is influencing hepatic lipid metabolism.

Efficacy of various doses of the two exemplary ASOs (ASO-2 and ASO-4) was also tested, including 3 mg/kg, 9 mg/kg, 27 mg/kg, and 81 mg/kg. The results are presented in a bar graph (FIG. 11A) and in a dose-response curve (FIG. 11B). Even lower doses of the ASOs were found to effectively suppress Dennd5b expression. For example, in view of the dose-response study, one of the exemplary ASOs (ASO-2) was identified as being capable of achieving about 95% knockdown of Dennd5b at a dose of 12 mg/kg.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Hubert, H. B., Feinleib, M., McNamara, P. M. & Castelli, W. P. Obesity as an independent risk factor for cardiovascular disease: a 26-year follow-up of participants in the Framingham Heart Study. Circulation 67, 968-977 (1983).
2. Sandfort, V. et al. Obesity Is Associated With Progression of Atherosclerosis During Statin Treatment. Journal of the American Heart Association 5 (2016).
3. Grundy, S. M. Obesity, metabolic syndrome, and coronary atherosclerosis. Circulation 105, 2696-2698 (2002).
4. Yanovski, S. Z. & Yanovski, J. A. Long-term drug treatment for obesity: a systematic and clinical review. JAMA 311, 74-86 (2014).
5. Iqbal, J. & Hussain, M. M. Intestinal lipid absorption. American journal of physiology. Endocrinology and metabolism 296, 94 (2009).
6. Levy, E. Insights from human congenital disorders of intestinal lipid metabolism. Journal of lipid research 56, 945-962 (2015).
7. Jones, B. et al. Mutations in a Sar1 GTPase of COPII vesicles are associated with lipid absorption disorders. Nature genetics 34, 29-31 (2003).
8. Marat, A. L., Dokainish, H. & McPherson, P. S. DENN domain proteins: regulators of Rab GTPases. J Biol Chem 286, 13791-13800 (2011).
9. Yoshimura, S., Gerondopoulos, A., Linford, A., Rigden, D. J. & Barr, F. A. Family-wide characterization of the DENN domain Rab GDP-GTP exchange factors. J Cell Biol 191, 367-381 (2010).
10. Su, A. I. et al. A gene atlas of the mouse and human protein-encoding transcriptomes. Proceedings of the National Academy of Sciences of the United States of America 101, 6062-6067 (2004).
11. Biesecker, L. G. et al. The ClinSeq Project: piloting large-scale genome sequencing for research in genomic medicine. Genome research 19, 1665-1674
12. Ye, Z., Kalloo, F. S., Dalenberg, A. K. & Kullo, I. J. An electronic medical record-linked biorepository to identify novel biomarkers for atherosclerotic cardiovascular disease. Global cardiology science & practice 2013, 82-90 (2013).
13. Das, S. et al. Next-generation genotype imputation service and methods. Nature genetics 48, 1284-1287 (2016).
14. McCarthy, S. et al. A reference panel of 64,976 haplotypes for genotype imputation. Nature genetics 48, 1279-1283 (2016).
15. Ishida, M., Oguchi, M. E. & Fukuda, M. Multiple Types of Guanine Nucleotide Exchange Factors (GEFs) for Rab Small GTPases. Cell structure and function, 61-79 (2016).
16. Chen, T. et al. Rab39, a novel Golgi-associated Rab GTPase from human dendritic cells involved in cellular endocytosis. Biochem Bioph Res Co 303, 1114-1120 (2003).
17. Lek, M. et al. Analysis of protein-coding genetic variation in 60,706 humans. Nature 536, 285-291 (2016).
18. Huan, T. et al. Dissecting the roles of microRNAs in coronary heart disease via integrative genomic analyses. Arteriosclerosis, thrombosis, and vascular biology 35, 1011-1021 (2015).
19. Vickers, K. C. et al. MicroRNA-223 coordinates cholesterol homeostasis. Proceedings of the National Academy of Sciences 111, 14518-14523 (2014).
20. Abdullah, M. et al. Peripheral blood gene expression profile of atherosclerotic coronary artery disease in patients of different ethnicity in Malaysia. Journal of Cardiology 60, 192-203 (2012).
21. Bennett, B. J., et al. Genetic Architecture of Atherosclerosis in Mice: A Systems Genetics Analysis of Common Inbred Strains. PLoS genetics 11(12), e1005711 (2015).
22. Lusis, A. J., et al. The Hybrid Mouse Diversity Panel: a resource for systems genetics analyses of metabolic and cardiovascular traits. J Lipid Res. 57(6):925-942 (2016).
23. Yu, R. Z. et al. Disposition and Pharmacology of Gal NAc₃-conjugated ASO Targeting Human Lipoprotein (a) in Mice. Molecular Therapy—Nucleic Acids 5, e317 (2016).
24. Aartsma-Rus, A. et al. Guidelines for Antisense Oligonucleotide Design and Insight Into Splice-modulating Mechanisms. Molecular Therapy 17, 548-553 (2009).
25. Integrated DNA Technologies. Designing Antisense Oligonucleotides (2011).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of treating hepatic steatosis, comprising:
identifying a subject having or at risk of developing hepatic steatosis, and
administering to the subject a DENND5B inhibitor, thereby reducing the expression of and/or activity of DENND5B in the subject.

2. The method of claim 1, wherein the DENND5B inhibitor comprise antisense oligonucleotide (ASO), CRISPR interference (CRISPRᵢ), miRNA, siRNA, locked nucleic acid (LNA) nucleotides, or a combination thereof.

3. The method of claim 2, wherein the inhibitor comprises a guide antisense strand consisting of about 10 to about 30 nucleotides.

4. The method of claim 3, wherein the guide antisense strand consists of about 10 to about 30 nucleotides consecutive nucleotides of SEQ ID NO: 1, or a complement thereof.

5. The method of claim 3, wherein the guide antisense strand consists of about 10 to about 30 nucleotides consecutive nucleotides within the first 2216 nucleotides of SEQ ID NO: 1, or a complement thereof.

6. The method of claim 1, and further comprising targeting the reducing the expression of and/or activity of DENND5B to the liver using a targeting moiety.

7. The method of claim 6, wherein the DENND5B inhibitor reduces the expression of DENND5B in the liver.

8. The method of claim 1, wherein the DENND5B inhibitor employs a nucleotide molecule targeting a protein coding region of DENND5B gene.

9. The method of claim 1, wherein the DENND5B inhibitor employs a nucleotide molecule targeting a non-coding region of DENND5B gene.

10. The method of claim 9, wherein the nucleotide molecule targets promoter of DENND5B gene.

11. The method of claim 9, wherein the nucleotide molecule targets promoter of DENND5B gene 3′ untranscribed regions (UTRs) of DENND5B gene.

12. The method of claim 1, wherein the subject is identified as having a risk of developing hepatic steatosis when the subject has diet-induced fatty liver disease; the subject has been identified as being at risk of developing diet-induced fatty liver disease; the subject has non-alcohol-induced fatty liver disease; the subject has been identified as being at risk of developing non-alcohol-induced fatty liver disease; the subject is obese as determined by BMI and/or abdominal circumference; the subject has type II diabetes; the subject has alcohol-induced fatty liver disease; and/or the subject has been identified as being at risk of developing alcohol-induced fatty liver disease.


SEQUENCE LISTING (15 sequences)

SEQ ID NO: 1 - DNA, 9506 bp, Homo sapiens
agacgcggcggctctcagctcgcgagcgcagctgcggctgcgagggcctctcaggccgacactccccccgcgcccctcccccacccggccgggctccggacccgccgcgcgcgagcccgcggccgcttcccccgcgccgagcgaggaggcaggctgcggcggcgagaggcggctgcgcggccatgggcgcgccagtgtcgccctagggcgggcgcacagaccacagccggagcggtggtggcagcctccgcagaaagccttcccgggcggcggcggcggcggcccctggagcagcgcggccgggatgagcgggagctgcgcggcgcccggcccgggctcgggctcctccccggccgcctgccgcttcgcgcactacttcgtgctgtgcgggatcgacgcggacagcgggctggagcctgacgagctggcgggcgaaaattttgaccagagtcctttgagaagaacattcaaatccaaagttctcgcccactatcctcagaatatagaatggaacccttttgatcaagatgcggtgaacatgttgtgcatgcctaaagggctatctttcaggacacaaacggacaataaagacccccagtttcactcatttataattaccagggaagatggttctcgcacctatggttttgttctcactttttatgaagaagttacaagtaagcaaatctgcacagcaatgcagacactttaccagatgcacaacgctgagcattacagcagtgtgtatgcttcatcttcctgcagtatggactcattggcaagtagtcttgatgaaggagatacaacttcccttttgaaactccagcgatacaactcctatgatattagcagagacaccctgtatgtttcaaaaagtatatgcttgatcacaccgttaccattcatgcaggcctgcaagaaattccttatccagctttacaaggctgttacctcacagcagccaccacccttgccacttgaaagctatatccacaatattctttatgaagtaccccttccacctccagggaggtcactgaaattttatggtgtttatgaacctgtcatctgccagaggcctgggcccagtgaactccccctctctgattacccccttcgggaggcatttgagctcctgggattagagaacctggtgcaggtgtttacctgtgttcttttagagatgcaaatccttctctactcacaagattatcaacgcctgatgactgtggcagaaggcatcaccacacttttgttcccatttcaatggcaacatgtttatgtgcccattctacctgcttctctgctacattttcttgatgctcctgtcccttatctgatgggccttcagtcaaaagaaggaactgaccgttctaaactagaacttcctcaagaggctaatttgtgttttgtggacattgacaaccattttattgagttgcctgaagaatttccacagttccccaataaagtggattttatccaagaactctctgaggttcttgttcaatttgggatccctcctgagggcagcctacattgcagtgagagtaccagcaaactgaagaatatggttctgaaagacttggtcaatgacaaaaagaacggcaatgtctgtactaataacatcagcatgtatgagttactgaagggcaatgaaaccatagcccgcttgcaggctctggccaagcgtactggtgtggctgtggaaaaaatggacctctctgcttctctgggtgaaaaagacaaggatttaaaactgcattgtgaagaggcagaactaagggactaccagctcaatgtacagctccgagaggtctttgctaaccgttttacacagatgtttgcagattacgaagcatttgtcattcagactgcccaggacatggaatcctggctgaccaaccgggaacagatgcagaactttgacaaagcttcctttctgtctgaccagcctgagccttacctgccatttctttcacgcttcattgaaacacagatgtttgccacctttattgataataaaattatgtctcagtgggaagagaaagatcctttgcttcgggtctttgacactcggattgataagataaggctgtataatgtaagggcacccaccttgcggacatctatatatcagaaatgcagcactttaaaagaagcagcccaatcaattgagcagagactgatgaaaatggatcacactgcaatccacccacatctacttgatatgaaaattggtcaaggcaaatatgagcaggggttctttccaaagttacagtccgatgtcttggcaacaggaccaaccagtaacaatcgctgggtaagtcggagtgccactgcacagcgcaggaaagaacgccttcgccagcattctgagcatgttgggctggacaacgacttgagggagaaatatatgcaagaggcacgaagtttaggaaaaaacctgaggcaacccaaactgtcagacctctctcctgcagttattgcacagaccaactgtaaattcgtagaaggcttattaaaagaatgtagaatgaagacaaagcgcatgttggtggagaagatgggacatgaagcggtggaacttggccatggagaagcaaacatcaccggcctggaggagaacaccttgatcgccagcctttgtgacctgctggagaggatatggagccatggcttgcaggtcaagcaggggaagtcggctttgtggtcacatttaattcaatttcaggacagagaagagaaacaagagcaccttgcagaatcaccagttgccctcggaccagaaagaagaaaatctgactcaggagttatgttgccaacgctcagggtctctcttattcaggacatgaggcatattcaaaacatgagtgagatcaagactgatgttggacgagctcgggcgtggataagactgtctctagaaaagaagctcttgtcccagcatcttaagcagttgctttctaaccaaccactcaccaagaagctttataagcgatatgcttttctacgttgcgaagaagaaagagagcagtttctttaccaccttctttctctcaatgctgtggactatttctgcttcaccagtgtgttcaccactatcatgattccgtataggtcagtgatcatcccaatcaaaaagctgagcaatgcaataatcacatcaaacccttggatctgtgtatcaggagagctgggagacacaggagtaatgcagattcccaaaaacctcctcgaaatgacctttgagtgccagaacttggggaagctgaccactgttcagattggtcacgataactcaggactgttagccaaatggctagtggattgtgtcatggtcagaaatgaaatcacaggacatacatacagattcccatgtgggcggtggctggggaaaggcattgatgatgggagcctggagagaattcttattggagagttgatgacatcagcatcagatgaagatctagtaaagcagtgtcggactccaccccagcagaagtcacccaccacggctaggagattgagcatcacttcactgacaggaaaaaacaacaaacccaatgctgggcagatacaagaaggaattggagaagctgtgaacaatattgtgaaacattttcataaacctgaaaaagagagaggaagcctcaccgtgttgctgtgtggagaaaatggcctggttgcagcccttgagcaagttttccaccatgggttcaaatctgcccgcatctttcacaagaatgtcttcatctgggacttcatagagaaagtggttgcttattttgaaacaactgaccagattctagataatgaagatgatgtccttattcagaaatcatcctgcaaaaccttctgccactacgtaaatgctattaatactgcacccaggaacattgggaaggatggcaaattccagattttagtttgccttggaacaagggatcgcctgctcccacagtggattccattgttagctgagtgtcctgccatcactcgaatgtatgaagagagcgctctcctgcgagaccgcatgactgtcaactcccttatccgaattctgcagaccattcaggacttcaccatagtcctagaaggatcactcatcaaaggagtggatgtgtaacccaactggctagaaactctcagtccaaaccttgctccttccccaactaggggaccgatttggacttgtctgacagtagtgagtcactgcaggggcagccaaacatatgccccatttggaacaatcctcactctacagacaaggcaaaatgttgtattgtagttcatttgaacctggaatttagtataaaatagagtattttcatgtgttagatgtgtgtaaatatgctatcttctttaagaaattatattactctaataagatgcttttcttagattgaatattcctgtgacttaaaataagtagcttaaaaatgttgggggttggggggaagagtggtgctagccaggaagaagccagtaaacatgtaaatatagtgcaacccagtcgggcttttttttttctccaggtatcacaaagaaacccagaatgcattgttatgtagtagccatccatcttggagtctgatcctttgtattgtggatgcacacagctttacctactgtactggtagtcttcacttctatcttttaagttacaaactgttttatatatttgaatcgctagtcacttttccagtcttttggaaaaaaatttcagtccgcacaagatgatataattggagtattttaaacactagaccccaggagcttggctgaactggaaagagaacatacaaagctctgagaaatgtgttcaattgtgggattataacttctttacacttcggaaagattagcctagactaacttcattcttggcatttttattcttgttatttgaggtactttgggaagatgtgaatttcagtgttagattcatagtgataagaactgcaaacagcaatttgattctgtacttttaatttggccatctaaactgtgcagtggtctacctcttaaggataaccatgagccctgtctttgttagggtctaatcacccacctctccagggcccatcccccctcgggaaatggaaggaatgtctaaggaggaggagggctgtgtgtggcttggaatgtttttgtgaaagcatttgttgaccatgataatgcaaataacagaaggagagagagaatgaaccctttctgggtgtttcagggcaagctttacgttcctttgtaggaaggggagtaataatgtaaaacaactgacccagagaagctaaaagccactggcagcacaggctgacaaaatggaagcaaagcaagaggtggcaacaattttgaaagaccaaaagttgtgagtaactttccagtttgatagtttataaagaagttaagtaaatgtagtattggctagttactgtaagtcctcatcttctggtaggtcctgtaactctaattaggcatttgtacatttttagcagcaaaattgcctcagcagagctcccagttttattcccagttgtggctgaaaagcaaagccatggcatcagtatccttgtgcaatcggaggttgctgggctttcaccacctgtgttgttaaccttatttcctggaggaagaaacagatgaaacaagtgcctaactccctttatcaaacacagccaaggacagcctcttttaacatgtgacttcatacttgaggaaaaggagagttgacagctgtatttaaaaacccatggagccgggtgcggtggctcacgcctatgatcccagcactttgggaggccgaggcaggcggatcacaaggtcaggagattgagaccctcctggctaacaccgtgaaaccccgtctctactaaaaatacaaaaaattagccgggcgtggtggcgggtgcctgtacaggaggctgaggcaggagaatggcgtgaacccgcgaggcggagcttgcagtaagctgagatcgcgccactgccctccagcctgggcaacagagcaagactccatctcaaaaaataaatagataaaaataaaaacccatggaaatggtttaagaaaatggttctgggtacaccagaaatttgtttgcctgaagctaaagtgtgtctacaatggtacagctcttccagggacatgggaagtttatcgtgggagatatccagaacctgttttacactgggctcctactgtgtttggtgatcagaaactatagaggggtccaacccaggatttcatgtttgtatcttcataaggccatgctcctgacaggtctaggcaacctgggggatgacttaggttaatgtgctaggattgacacacctcatccagccttcttactttgtggatgggaacacttaaggaatagagagattaaatcttacacttaccccacagctggttactgacaacacaagctctagaattcatgtttgtttgtttgttttatttcaccaggctgcatgagactgaccttccggcattcttactaatagtctttctattatactattggatctttattcatccacctatacaagtaggaaataaggatgaatattcatttagaagaattaatattcatattaacacattgctctcagattattgggaattaacatcttgtaaaaactgggagcaggccaggtacggtggctaacgcctgtaatcctgacactttgggagtctgaggcgggtggttcatctgaggtcagtcgggagttcaagaccagactggccaacatggtgaaaccccgtctctactaaaaaaaaaaaatagacaaaaattagcgggggggtggtggcagggcctgtaattccagctactcaggaggctgaagcaggagaatcgtttaaacacaggaggtggaggttgcagtgagccgagattgtgccactgcactccaacctgggtgacaagagcgagactccgtctcaaaaaaaaaaaaagaaaaaaactgcagccaaagttggatttgcggcctatgaataaaaccattactttaaaaagtaatacccccaaaaaagttacacctgaagtttcatcttcatgcctttgtgtaatattactataatggtgaaatgatgatttaaaaaaaacacatcagaactgacacatcctcttggatgatgggtccttcagtcaccttgtgagcccatgtacttattccgttgatacagcctgtgctataaacatgtggggccctccttgtgtgagctgcttgaatggccagtacccccattcttttgaatattcccaatggcagtcaatcttggcctttaagaataaatttaaatatttggaaatagtccttaggatccaagtttgatcaatgaaaaggatgcttaagctgagtttggggtcacataagaagtgcagttataaagaaataagacatgttgggcatggcagtacacacctgtagtcccagctgcttgggaagccaaggcaggaggatcacttcaacccaggagtttgagtccagcctgggtaacatagggaaaccccatctcaaaaaaacaaaacaaaggacttagtttttatgtggctcataagtgtctctaagttataccaaaagtttgagcacaacagtagtagcagaatgcttagaataaatgcttgacttttaagctgacttttggaaggaccatacccacatagaggcatcttttctgctgtggtaattttttaaattaatacattgctttaggattacatctcaaagtaaataaaaaaatcaatatttaatttgtttttttcctgcaaataaattaaatgctgggaccataatgattatttttgcatagtaatagctggtgaagaagttgttttaaaattaaattgccaaaagtattacctcttgaaaaatttattttcccaaagtttttcatatgtcatgaaacccctaatcttgacctttatgacacagtggcattacactgatgttggaagcatgtgtgagatctgtgccctggtcgggagtcatatgccttaaaaatgactgcgagtcagtgtgaagtagtataaagtggagtcctacgtgggaaaaagcaggctactgaggacaaaaacctcccaaccctccccaccttgttttccactgggaatgagaatttgatatgagaaacttcaaccttgcagaagtgtggatttccctctgaaatttaaatgtttcacacatttttgtaccttttgtaaccttgtggtatttactttcacatggctttttaaaaaattattttaatgaacttctatcacactatatgaatgatatgggggaaatactgaactgctcatatttccttttttggcaataaagcactgttatatgtaaatgtaagtgaaaaagaggttcaagactatacaacagcttatgtgtagtatatgttgacatccctaaggtattttgaaacatgaggtgacagacaaggacctcaaatttccagggaaatatttatttcacaacattaggagtgtagctcttcataatgaaatatcttctttctgctaaagattacaatttttcatttagggaactgtgaatattttctgtgaggtttacagaaactagtgtatccttttcactatgttgaaaagacaatctgaactttagctatttatatttcagtattaacacaccttcactgattgagagtcttgagcccttctctacttttataaagtgaatcctatttccaagtcatccatgtcttttgaagtctgtttcttatataagtatttattactttactgtttagcataaataggagacagaaaaggataataaactactaggtcctttattcctattaaaataaatttataaatcccttgaatcaactgaattctaagctttttgaagaggttgctgatgtagtgtgatcaaaagaatctaaggagaacctcctctccaaaatctgattggagctgttcaccctctcaaggacaagataaagattgacttcagagatatatcacatcaagctagaggaaaaaaaaaaaacctcaaataaaatttttcctcattcgaagggatccagggtatccttgggttatgtaggagggaatcatatttgttctaatttacctgaatgtacttgtgtgtgaatccaatcttgattctaaacaccaaatcttgtcaacagtagagaagtatagtaaggagttcctgttgtcaatcaaatgagggtgtgaaacatttagaatgctctgtgttacacttgaaccatgtataaatgtaacctgtgtatcttttttttaattagcttttctgaatttaaattatattgtttttctttgtattgttagcaaaatgcatcagaactggattttttttctcatctgaacataatataaaatttgaaagagtattgtgatattaagcactttaacatacatatcagacaaactgacgtgggtttttcaggtttgggagtacatttaaatatgacttttcatgctttgttctttacaaataaaactgtgggattgata

SEQ ID NO: 2 - DNA, 39 bp, Mus musculus
cttatctgatgggtcttcagtcaaaagaaggaactgacc

SEQ ID NO: 3 - DNA, 20 bp, Mus musculus
cttatctgatggaactgacc

SEQ ID NO: 4 - DNA, 30 bp, Artificial Sequence
  oligo
ccagtgtcgccctagggcgggcgcacagac

SEQ ID NO: 5 - DNA, 30 bp, Artificial Sequence
  oligo
atgaagaagttacaagtaagcaaatctgca

SEQ ID NO: 6 - DNA, 30 bp, Artificial Sequence
  oligo
ttcatcttcctgcagtatggactcattggc

SEQ ID NO: 7 - DNA, 30 bp, Artificial Sequence
  oligo
ttaccattcatgcaggcctgcaagaaattc

SEQ ID NO: 8 - DNA, 30 bp, Artificial Sequence
  oligo
gctgttacctcacagcagccaccacccttg

SEQ ID NO: 9 - DNA, 30 bp, Artificial Sequence
  oligo
cccttccacctccagggaggtcactgaaat

SEQ ID NO: 10 - DNA, 30 bp, Artificial Sequence
  oligo
cctgtcatctgccagaggcctgggcccagt

SEQ ID NO: 11 - DNA, 30 bp, Artificial Sequence
  oligo
gagaacctggtgcaggtgtttacctgtgtt

SEQ ID NO: 12 - DNA, 30 bp, Artificial Sequence
  oligo
cctgtgttcttttagagatgcaaatccttc

SEQ ID NO: 13 - DNA, 30 bp, Artificial Sequence
  oligo
atagcccgcttgcaggctctggccaagcgt

SEQ ID NO: 14 - DNA, 30 bp, Artificial Sequence
  oligo
gcatttgtcattcagactgcccaggacatg

SEQ ID NO: 15 - DNA, 30 bp, Artificial Sequence
  oligo
ataaggctgtataatgtaagggcacccacc